(19) United States
(12) Patent Application Publication
(10) Pub. No.: US 2024/0138689 A1
(43) Pub. Date: May 2, 2024

(54) Vascular Pressure Measurement Systems and Methods Including Vascular Pressure Differential Diagnostic Systems and Related Methods

(71) Applicant: VahatiCor, Inc., Los Gatos, CA (US)

(72) Inventors: Kevin H. Van Bladel, Livermore, CA (US); Marwan Berrada-Sounni, Los Gatos, CA (US); Scott Edward Parazynski, Houston, TX (US)

(21) Appl. No.: 18/279,947

(22) PCT Filed: Mar. 1, 2022
(86) PCT No.: PCT/US22/18253

§ 371 Date: Sep. 1, 2023

Related U.S. Application Data
(60) Provisional application No. 63/155,122, filed on Mar. 1, 2021.

(51) Int. Cl. A61B 5/0215; A61B 5/00

(52) U.S. Cl. CPC A61B 5/02158; A61B 5/6851; A61B 5/6853

(57) ABSTRACT

Systems and methods for pressure differential measurement and hemodynamic response assessment in vascular lumens are disclosed. Systems include catheter-based devices with variable flow restriction to create a controlled variable pressure differential (ΔP) within the coronary sinus. Methods are disclosed that allow a range of pressure changes to be mapped to a range of flow restriction diameters on a patient-specific basis as well as mapped to clinically measurable indicators of restriction/pressure change effect on hemodynamic and cardiac response.

### RELATED APPLICATION DATA

This application claims the benefit of priority of U.S. Provisional Patent Application Ser. No. 63/155,122, filed Mar. 1, 2021, and titled “Pressure Measurement System and Method for Adjustable Vascular Venous Reducer as a Hemodynamic Modifier”, which is incorporated by reference herein in its entirety.

### FIELD OF THE DISCLOSURE

The present disclosure generally relates to the field of vascular and cardiac pressure differential diagnostic systems and methods. In particular, the present disclosure is directed to vascular pressure differential diagnostic systems and methods and including an adjustable and controlled vascular occlusion device, allowing an in-vivo variation of hemodynamic restrictions for assessing physiological patient response thereto.

### BACKGROUND

Occlusion of the coronary sinus has been studied for controlling the flow within the vasculature after ST-segment elevation myocardial infarction (STEMI). Studies in patients of intermittent coronary sinus occlusion following a STEMI has shown promise in reduced infarct size attributed to re-distribution of the flow to the deprived perfusion border zones and increased collateral flow. Similar to permanent occlusion of the coronary sinus, the flow also can be reduced by placing a temporary vascular restrictor within the coronary sinus to generate a body physiological response. A flow/pressure modulator is a vascular restrictor used by physicians to modulate hemodynamic flows and pressures inducing an artificial physiological effect beneficial to the patient. The vascular restrictor causes a pressure differential (AP) within the coronary sinus to partially block the venous outflow which improves flow to the arterial system and collateral flow. Examples of flow/pressure modulators providing a vascular flow restriction or disclosed, for example, in the present Applicant's co-pending International Application, Publication No. WO 2021/226014, entitled “Vascular Flow and Pressure Modulator,” which is incorporated herein in its entirety.

Patients have seen an improvement in angina pain symptoms when implanted with a vascular restrictor. However, the size of the restrictor is not well understood. The specific diameter restriction necessary can be personalized to the patient's response to the ΔP within the coronary sinus. There is a need to measure the ΔP to determine which patients could benefit and the amount of restriction. This device provides a means to measure this ΔP and adjust the restriction before implanting the patient. The application of such a device is broad to all types of vessels (arterial and venous), and more particularly for reducing flow and augmenting pressure in the coronary sinus to benefit refractory angina patients.

A coronary sinus flow/pressure modulator is a device to aid in managing patients with angina refractory to optimal medical therapy and not amenable to further revascularization. The device is a controllable flow-limiting scaffold providing a hemodynamic restructure within the coronary sinus lumen. The intention is to increase back pressure within the coronary sinus to drive higher perfusion to the distal coronary bed and redistribute trans-myocardial blood flow. Many such devices are formed by a porous scaffold that endothelializes to create a reduced diameter orifice. However, until the scaffold is entirely or close to fully endothelialized, the potential therapeutic effect may not be realized. It, therefore, can be difficult to ascertain if a patient is benefiting from such treatment, and there is a need to disclose a device that would temporarily mimic the flow modification that a patient would experience in order to determine if the patient would be a responder or not to the permanent therapeutic device.

### SUMMARY OF THE DISCLOSURE

In one implementation, the present disclosure is directed to a vascular pressure differential diagnostic system that includes a catheter having proximal and distal ends, the distal end configured for positioning within a patient's vasculature at a pressure monitoring site; a variable flow restrictor disposed adjacent the distal end of the catheter; a first pressure sensor disposed distally with respect to the variable flow restrictor; and a second pressure sensor disposed proximally with respect to the variable flow restrictor, whereby a pressure differential between the first pressure sensor and second pressure sensor is measurable and mappable to varying flow restrictions.

In another implementation, the present disclosure is directed to a method for determining hemodynamic and cardiac response to vascular flow restriction. The method includes measuring a base line pressure at a monitoring site within a patient's vasculature; partially occluding the vascular lumen at the monitoring site with a plurality of differently sized flow restrictions; measuring a pressure differential across the partial occlusion for each differently sized flow restriction; and identifying a flow restriction size corresponding to a desired pressure differential.

In yet another implementation, the present disclosure is directed to a method for determining hemodynamic and cardiac response to vascular flow restriction. The method includes positioning a vascular pressure differential diagnostic catheter within a vascular lumen at a monitoring site; partially occluding the vascular lumen to create a first size flow restriction at the monitoring site with the vascular pressure differential diagnostic catheter; measuring a pressure differential across the partial occlusion using pressure sensors disposed on the vascular pressure differential diagnostic catheter upstream and downstream from the partial occlusion; repeating the partially occluding and measuring steps with at least a second size flow restriction, the measuring step performed for each different sized flow restriction; monitoring patient physiological response to each different sized flow restriction of the partial occlusion; evaluating patient physiological response to each different sized flow restriction based on predetermined physiological response criteria; selecting as a candidate for an implanted flow restricting device the flow restriction size corresponding to the predetermined physiological response criteria; and identifying the flow restriction size corresponding to a desired pressure differential.

### BRIEF DESCRIPTION OF THE DRAWINGS

To illustrate the disclosure, the drawings show aspects of one or more embodiments of the disclosure. However, it should be understood that the present disclosure is not limited to the precise arrangements and instrumentalities shown in the drawings, wherein:

FIG. 1 schematically depicts a vascular pressure differential diagnostic system according to the present disclosure disposed of in the coronary sinus.

FIG. 1A schematically depicts an alternative embodiment of a vascular pressure differential diagnostic system according to the present disclosure using a plurality of flow restricting balloons of different diameters.

FIG. 1B schematically depicts another alternative embodiment of a vascular pressure differential diagnostic system according to the present disclosure with an additional, distally disposed full occlusion balloon.

FIG. 2 is a cross-section view through section line A-A of the vascular pressure differential diagnostic system shown in FIG. 1.

FIG. 3 schematically depicts a proximal hub arrangement for vascular pressure differential diagnostic systems disclosed herein.

FIG. 4 schematically depicts an alternative embodiment of a vascular pressure differential diagnostic system according to the present disclosure.

FIG. 4A schematically depicts another alternative embodiment of a vascular pressure differential diagnostic system according to the present disclosure.

FIG. 5 is a cross-section view through section line B-B of the vascular pressure differential diagnostic system shown in FIG. 4.

FIG. 6 is a schematic cross-section of a further alternative embodiment of a pressure measuring system.

FIG. 7 is another schematic cross-section of the embodiment shown in FIG. 6 in a different state of operation.

FIG. 8 is a flow diagram illustrating an embodiment of a method for monitoring vascular pressure according to the present disclosure.

FIG. 9 is a schematic block diagram depicting a vascular pressure diagnostic system according to an embodiment of the present disclosure.

### DETAILED DESCRIPTION

To assist in providing vascular pressure modulating therapy customized to the patient's hemodynamic environment, embodiments disclosed herein provide temporary restrictions allowing evaluation of the hemodynamic gradient or ratio to determine each patient's desired reducer diameter. Using prior systems, coronary sinus reducer procedures are often performed with less than full information regarding patient-specific hemodynamic response. Systems and methods described herein may be employed venously or arterially to best evaluate which patients would most benefit from treatment and at what size orifice for improved patient response. Systems and methods described herein also may be used in other areas to determine effective treatments, for example, in other vascular applications such as Arteriovenous malformations, (AVMs), Arteriovenous fistulas (A-V fistulas), aneurysms, and other neonatal cardiovascular anomalies.

Embodiments disclosed herein thus address potential concerns regarding less than complete hemodynamic response information by deploying a personalized and variable restriction to maintain a certain pressure gradient. Pre-procedural evaluation using a temporary catheter provides information about which patients will benefit and the restrictor's correct size. Monitoring and maintaining such restrictions are also elements of disclosed systems and methods. Monitoring may be provided through sensors, either passive or active (e.g., active requiring a power source such as a battery). Depending on the hemodynamic response, the restriction could be adjusted automatically in-situ through an external stimulus or internally through a simple procedure (e.g., angioplasty balloon). Aspects of the present disclosure thus include but are not limited to pre-procedural hemodynamic gradient measurements to determine optimum restrictor size, methods and systems to measure pressure gradient in patients to personalize therapy, and patient monitoring using either active or passive hemodynamic-based instruments.

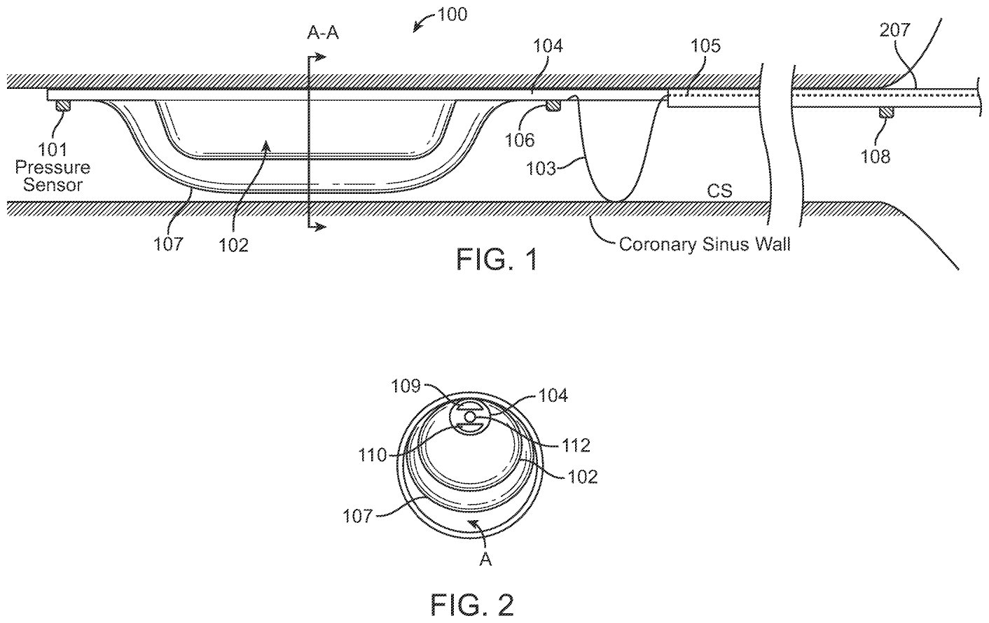

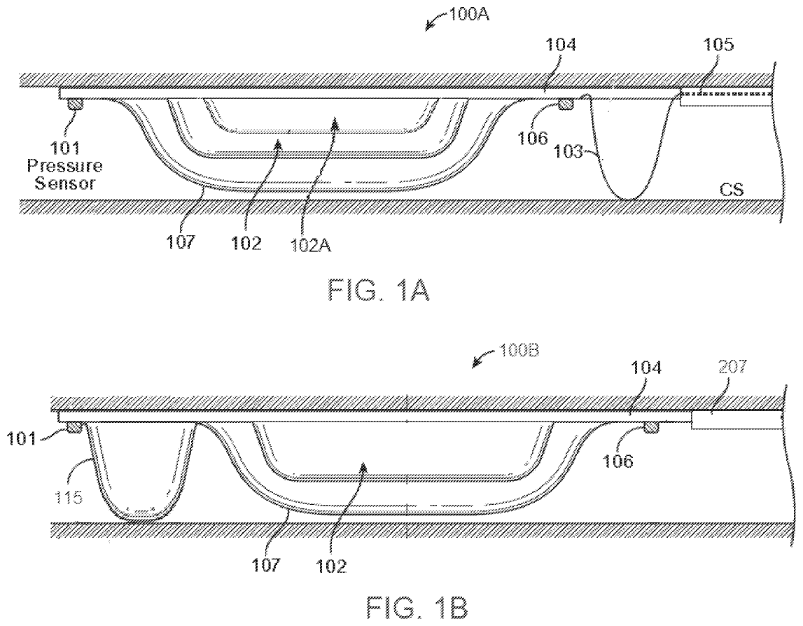

As illustrated in FIGS. 1 and 2, in one embodiment of the present disclosure, vascular pressure differential diagnostic system 100 includes catheter body 104, which may be a triple lumen catheter as shown in FIG. 2, or a double lumen catheter in other embodiments. In the illustrated embodiment, catheter body 104 is provided with two inflatable balloons, inner non-compliant balloon 102 and outer compliant balloon 107. Inflation lumen 110 communicates with inner non-compliant balloon 102 and inflation lumen 109 communicates with outer compliant balloon 107. Central guidewire lumen 112 provides a pathway for a guidewire to facilitate placement. In some embodiments, a single balloon, compliant or non-compliant, may be used. Each inflation lumen of catheter body 104 is used to inflate and deflate balloons 102 and 107 in a conventional balloon catheter manner. In this embodiment, catheter body 104 and balloons 102, 107 are configured to be eccentrically positioned within the vascular lumen in which they are deployed. The coronary sinus (CS) is depicted in FIGS. 1 and 2, but other vascular lumens may be monitored in the same manner. Anchor wire 103 is configured to temporarily fix the distal end of catheter body 104 and balloons 102, 107 at a monitoring site and to maintain them pushed up against the lumen wall at one side of the vascular lumen. Anchor wire 103 is deployed and retracted via control wire 105. Catheter body 104 and anchor wire 103 are delivered and retrieved from a monitoring site in the vascular lumen via a sheath or guide catheter 207. Multiple pressure sensors may be included in system 100. In the disclosed embodiment of FIG. 1, three pressure sensors are provided: pressure sensor 101 at the distal end of catheter body 104, pressure sensor 106 at the proximal sides of balloons 102 and 107, and pressure sensor 108 more proximally disposed on a distal end of guide catheter 207. Pressure sensor 108 is disposed to allow for more proximal sensor measurements, such as in the right atrium (RA) when system 100 is deployed in the coronary sinus (CS).

With the embodiment shown in FIGS. 1 and 2, inner, non-compliant balloon 102 provides a fixed-sized vascular restriction when inflated. Outer compliant balloon 107 can be inflated at varying sizes to increase the vascular occlusion up to a full occlusion, depending on the type of pressure measurements to be taken. Additional overlapping balloons may be provided as described below. The area restricted flow passage (A) may be determined based on the relative cross-sectional areas of the balloons and vessel lumen. Thus, the reduced cross-sectional flow area (A) may be calculated as:

A=πrᵥ²−πr_{b}² [1]
where: rv=vessel inner radius
   rb=inflated radius of occlusion balloon
      For example, using a flow-restricting balloon inflated to a diameter of 6 mm within an 8 mm diameter vessel, the restricted cross-sectional flow area will be 7π mm² (approximately 21.99 mm²). If it is determined that the ΔP measured between pressure sensors 101 and 106 is at a clinically desired level, then this information may be used to select a CS reducer device with the same internal flow restriction, in other words an internal orifice with a diameter of approximately 5.29 mm.

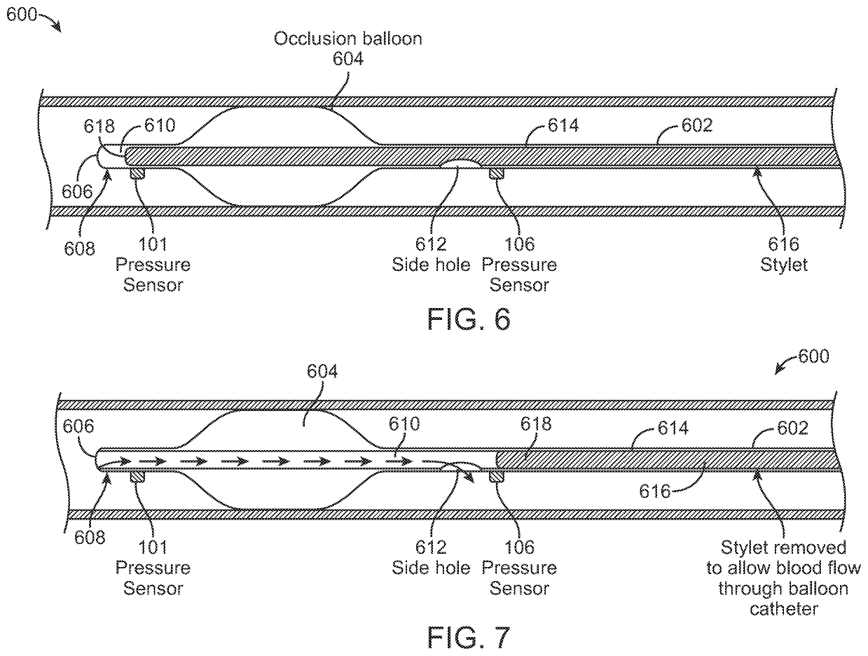

As will be appreciated by persons of ordinary skill, based on the teachings of the present disclosure any number of nested restricting balloons may be provided, subject essentially only to manufacturability constraints. For example, as shown in FIG. 1A, catheter system 100A is provided with three nested balloons, 102A, 102 and 107. Additional balloon 102A may be configured as a resilient or non-compliant balloon. When a plurality of non-compliant balloons are used, the device could be used to establish predetermined flow restriction steps without precise pressure control. In another alternative, shown in FIG. 1B, catheter system 100B includes a separate full occlusion balloon 115 that is disposed distally with respect to the flow restricting balloons 102 and 107. In such embodiments with additional balloons, additional inflation lumens may be provided within catheter body 104 to permit individual control of each balloon. In a further alternative embodiment, guidewire lumen 112 may also serve as blood flow passage with a variable restriction provided by a side port, such as side hole 612 as shown in FIGS. 6 and 7. In this case, the open distal end of guidewire lumen 112 serves as an entry port and the guidewire itself, or a separate flow control stylet, such as stylet 616 also shown in FIGS. 6 and 7, may be used to fully or partially obstruct the side port to provide a variable flow restriction as described below in connection with system 600.

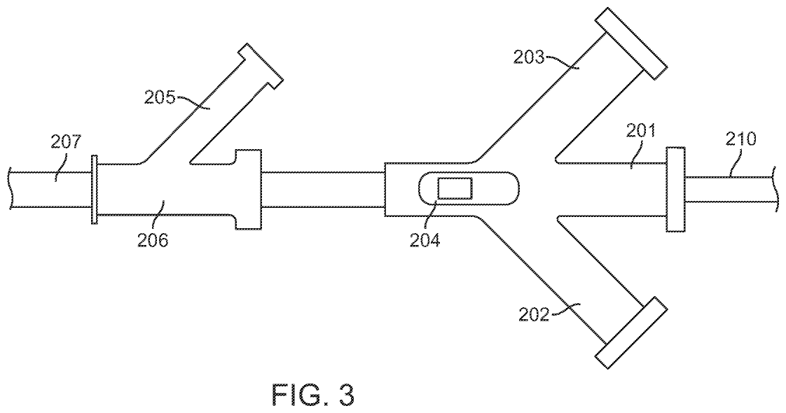

FIG. 3 shows one embodiment of a control hub system for a vascular pressure differential diagnostic system as disclosed herein. As shown herein, hub 206 provides for control of guide catheter 207. Port 205 may be configured as an additional inflation port to control inflation and deflation of an additional balloon(s) outside of guide catheter 207, which can be inflated to obtain wedge pressure in the coronary sinus (for example as shown in FIG. 1). Hub 201 controls catheter body 104 and includes inflation ports 202 and 203 for controlling inflation and deflation of balloons 102, 107 via the dual lumens of the catheter body 104. Hub 201 also provides a central lumen port to receive guidewire 210 to facilitate navigation of guide catheter 207 to the vascular monitoring site of interest. With guide wire 210 removed, the central lumen port may be used as a further pressure monitoring port. Control mechanism 204 controls deployment and retraction of anchor wire 103 via control wire 105.

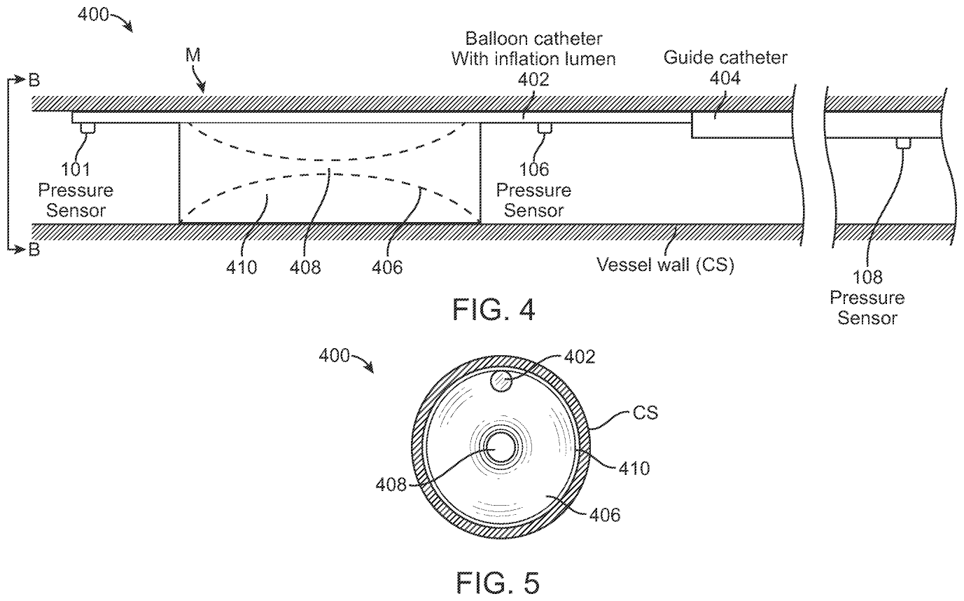

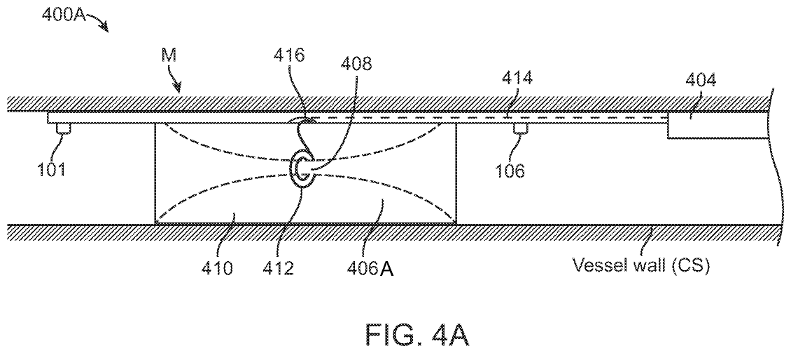

An alternative vascular pressure differential diagnostic system 400 is shown in FIGS. 4 and 5. In this embodiment, a catheter body 402 is delivered through a guide catheter 404 to the vascular lumen monitoring site (M), which may in some embodiments be the coronary sinus (CS). A balloon 406 configured to provide an internal hour-glass shaped orifice 408 is provided in the distal region of the catheter body. The balloon 406 may be inflated and deflated through a central inflation lumen (not shown) in the catheter body 402, which inflation lumen may be configured in one example as described above. Because the hour-glass shaped balloon 406 is configured to contact the vessel wall around its periphery 410 with the orifice restriction 408 centrally located, a separate anchor wire may not be required in this embodiment. Alternatively, an anchor wire as described hereinabove may be included for added anchoring security. Pressure sensors 101, 106 and 108, such as shown and described for the embodiment of FIG. 1, also may be employed in the embodiment of FIG. 4.

Balloon 406 may be configured in a number of alternative structures to provide a controllable orifice 408. For example, balloon 406 may be formed with a non-compliant outer wall such that the balloon would be provided in a number of sizes selected to match the inner diameter of a particular monitoring site in the vasculature. In this alternative, the inner wall of the balloon forming orifice 408 is formed from a resilient material so that inflation pressure can be used to change the orifice diameter without changing the outer diameter of the balloon. In a further alternative, as illustrated in FIG. 4A, system 400A includes internally hour-glass shaped balloon 406A with a cinch 412 to provide adjustment of the size of controllable orifice 408. Cinch 412 is controlled by control wire 414, which passes through internal inflation port 416 and traverses the inflation lumen 402. In one embodiment, cinch 412 is biased towards a maximum open state whereby tensioning of control wire 414 decreases the orifice area from its maximum to increase the flow restriction in a predictable relationship to longitudinal movement of the control wire at the system hub. Enlarging the orifice in such embodiments is accomplished by releasing tension on the control wire to reduce the flow restriction, again in a predictable relationship to longitudinal movement of the control wire.

In a further alternative embodiment, system 600, shown in FIGS. 6 and 7, has a catheter body 602 including a distally positioned occlusion balloon 604. An inflation lumen (not shown) for inflating the balloon is provided in the catheter body as previously described. At the distal tip 606 of the catheter body 602, distal to the occlusion balloon 604, a central blood flow port 608 is provided and defines the entry to a reduced orifice diameter flow passage 610. A side hole 612 proximally disposed with respect to the occlusion balloon 604 provides the exit port for the reduced orifice flow passage 610. The catheter body 602 also defines a central lumen 614 for a retractable stylet 616 that can be used to allow access to or close the reduced orifice diameter flow passage 610. With the stylet 616 fully extended distally, the entry port 608 and flow passage 610 is blocked entirely. Retracting the stylet distal end 618 to a position proximal of the side hole 612 allows maximum flow through the reduced orifice diameter flow passage 610. Alternatively, the position of the stylet distal end 618 may be advanced somewhat distally from the fully open position shown in FIG. 7 so as to further reduce flow through the flow passage 610 by partially obstructing side hole 612. As in other embodiments, distal and balloon proximal pressure sensors 101 and 106 may be included. A further proximal pressure sensor, positioned for example as pressure sensor 108 in FIG. 1, may be employed.

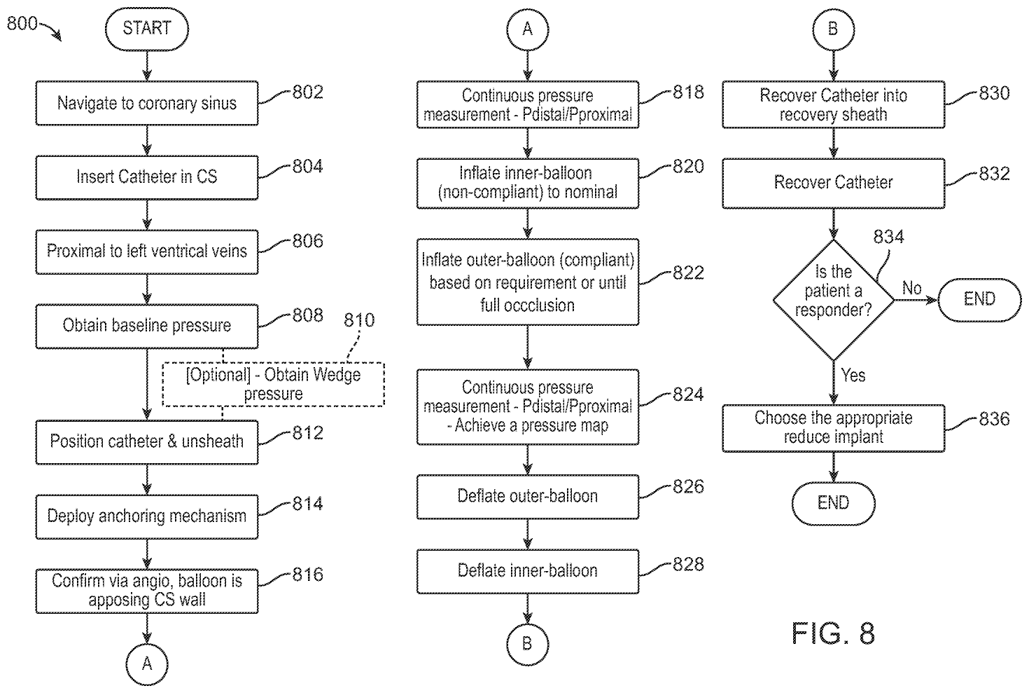

According to the present disclosure, FIG. 8 illustrates one example of a method of pressure monitoring and hemodynamic response assessment using a double balloon device such as shown in FIGS. 1 and 2. Disclosed method steps allow for the determination of patient response and selection of appropriate restriction orifice size before implantation of a coronary sinus reducer device is performed. In this example, as shown in FIG. 8, the device is navigated to coronary sinus 802 and then inserted into the coronary sinus 804. In some embodiments it may be preferable to further position the device at a monitoring site proximal to left ventricle veins 806. Once positioning at the selected monitoring site is confirmed, a baseline pressure is obtained before inflation of the balloon 808. At this point it may be desirable to optionally obtain wedge pressure 810. Alternatives for obtaining wedge pressure include conventional wedge pressure measurement using a separate Swan-Ganz catheter in the pulmonary artery. Alternatively, an occlusion pressure within the coronary sinus may be obtained by fully occluding the coronary sinus using one of the alternative devices described hereinabove. The occlusion pressure in the coronary sinus may be used as a baseline occlusion pressure as well as baseline open pressure.

Catheter position can be again confirmed and then the catheter unsheathed 812. The anchoring mechanism, if used, is then deployed 814. Using appropriate visualization, such as angiography, balloon apposition with the coronary sinus wall is confirmed 816. Once positioning of the pressure measurement device is confirmed, desired pressure measurements may be taken. The foregoing positioning steps provide an illustration of the positioning of a device such as system 100 shown in FIGS. 1 and 2. Other embodiments disclosed herein may employ variations in the positioning process as will be apparent to persons of ordinary skill based on this example and the further teachings of the present disclosure. Similarly, the following description of pressure measurement steps is based on an example using system 100. Pressure measurements taken with other embodiments disclosed herein will vary in process according with their structures as described herein above.

Once positioning of the device at the monitoring site is satisfactorily confirmed, continuous pressure measurement is commenced 818. This pressure measurement includes at least continuous measurement of pressures P_{distal} and Pₚᵣₒₓᵢₘₐₗ (for example via pressure sensors 101 and 106, respectively). The flow restriction is then initiated by inflation of inner-balloon (102 non-compliant) to nominal size 820. Thereafter inflate outer-balloon (107 compliant) based on specifically identified restriction requirement or until full occlusion 822. Simultaneously with outer balloon inflation, continuously measure pressures P_{distal} and Pₚᵣₒₓᵢₘₐₗ to define a restriction vs ΔP map 824.

In a further alternative embodiment, at this stage or another convenient process step, the ΔP can also be corelated to reduction in ST segment changes and/or improved cardiac output measured by intracardiac sensors, nuclear perfusion scanning, or by ultrasound. For example, when the lumen is wide open the baseline contractility or ST elevation can be monitored, and when the balloon is inflated within the vessel the internal diameter will narrow. The baseline contractility and ST segment can be observed to see a substantial improvement in cardiac performance. If the vessel is narrowed too far, these physiological parameters (ST segment elevation, CO, rate/rhythm) would get worse. In this manner a three-dimensional pressure map can be formulated with ΔP vs. restriction vs. cardiac status indicator (i.e., ST segment or other indicators mentioned above). Therefore, the ideal restriction can be determined, potentially with greater accuracy, by this technique. In another alternative, where clinically appropriate, an awake patient may be queried as to level of angina pain as the ΔP is altered via changes in the restriction diameter or flow in real-time.

Once the desired pressure measurements are complete, the device may be removed. Exact removal steps will depend upon the particular pressure measurement device used. For example, again with reference to system 100 of FIG. 1, and as shown in FIG. 8, the first step of removal may be to deflate outer balloon 826. Next, the inner balloon is deflated 828. Thereafter, recover the catheter into recovery sheath 830 and recover the catheter 832. At any point after pressure measurement and optional creation of the pressure map, the patient can be determined to be a candidate for a permanent coronary sinus reducer implant 834 and the appropriate diameter/orifice size implant selected 836. The selected implant may be configured and implanted as described in Applicant's aforementioned co-pending and incorporated application, Pub. No. WO 2021/226014.

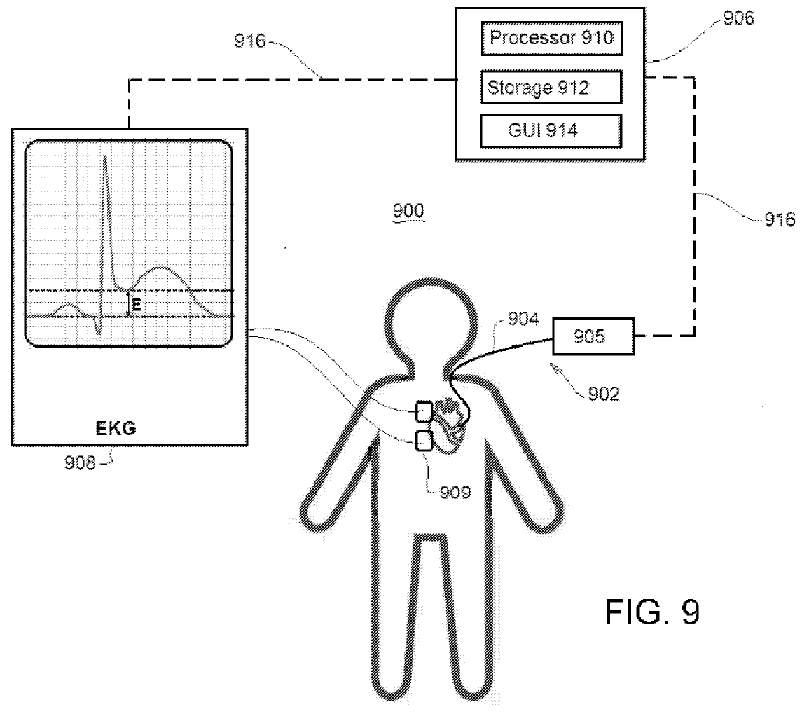

Pressure measurement catheter systems as disclosed herein may be incorporated into pressure mapping diagnostic systems to provide pressure mapping as described herein as a partially or fully automated function. An example of such an automated pressure mapping system is system 900, shown schematically in FIG. 9. System 900 includes pressure differential diagnostic system 902, which may comprise any of catheter based systems 100, 400 or 600 or further alternatives of those systems as taught in the present disclosure. System 900 also may optionally employ other types of pressure differential monitoring devices as may be determined appropriate by persons of ordinary skill based on the teachings of the present disclosure. Other primary components of system 900 include control unit 906 and a cardiac monitoring system such as EKG system 908, which uses patient electrodes 909 to monitor cardiac rhythm and produce an electrocardiogram. Components of system 900 communicate with control unit 906 via communications links 916, which may be wired or wireless links used in conventional communications protocols.

Control unit 906 generally comprises one or more processors 910, a storge module/memory 912 and user interface 914 as well as any other required hardware to process, received and send appropriate control and data signals via communications links 916. Control unit 906 may be programmed by persons of ordinary skill in the art based on the teachings of the present disclosure to automatedly control and monitor balloon inflation, determine restriction size and measured ΔP, and to correlate restriction size, ΔP and, where desired, electrocardiogram features such as ST segment elevation (E) to produce pressure maps as described hereinabove.

Further features and advantages of embodiments disclosed herein include:
A balloon-based catheter can be inflated within the coronary sinus to mimic the hemodynamic restriction that a permanent implant would provide.
A balloon-based catheter with single or multiple overlapping balloons can be inflated independently from each other to provide coarse or fine inflation and thereby precise control of the diameter of the temporary flow restriction.
A balloon-based catheter with single or multiple overlapping balloons made of the same or different balloon material.
As described above, a balloon-based catheter may be located in the coronary sinus with a bias against the vessel wall.
A balloon-based catheter is a multi-lumen catheter with 2 balloons with an anchoring mean with 2 pressure sensors, one on the distal end of the balloon and the second is on the sheathing-tube. The balloons are located in two regions. Two concentrically constructed balloons provide a coarse and fine inflation control to occlude the vessel, and the other is on the sheathing-tube.
A temporary balloon may also contain lumens to allow for pressure monitoring on either side of the hemodynamic restriction.
An anchor located either in the balloon's distal or proximal ends would ensure immobility of the catheter during balloon inflation.
A balloon-based catheter comprising two overall elements: a balloon catheter and a sheathing-tube to cover the balloon and collapse the anchor.
A balloon-based catheter may comprise a folded balloon with an anchor that is controlled proximally with a push/pull, a rotating mechanism, or a combination of the two.
A pressure sensor may be placed on the distal end on a guide catheter or sheath tube, providing a continuous right atrium (RA) pressure measurement.
Three pressure sensors may be placed and utilized as follows: One on the distal end of the balloon catheter (P1), proximal to balloon (P2), and the last one on the distal end of the sheathing tube (P3). P1 is measuring P_{distal}, P2 is measuring Pₚᵣₒₓᵢₘₐₗ, and P3 is measuring P_{RA}.
The proximal end of the catheter may be provided with three ports and a slider. The three ports may be configured as guidewire lumen/pressure monitoring, non-compliant balloon inflation port, and compliant balloon inflation port. The slider allows for adjustment and expansion of the anchor against the vessel wall. When pushing to its resting position, the slider shall completely collapse the anchor.
The hub has one port for inflating a compliant balloon to measure wedge pressure in the coronary sinus on the guide catheter or sheath tube.
As described herein, a balloon catheter may provide information regarding what permanent size restriction is necessary for each patient.
Balloons for systems described herein may be made of silicone or polyurethane or any other commonly used balloon materials in the art.
With calculations using the vessel size compared to the balloon diameter, the overall volume reduction can be achieved to determine the optimum restrictive implant.

Embodiments of the present disclosure provide several advantages over existing technologies or solutions. These advantages may include, but are not limited to:
The restriction of flow is measured using a temporary restrictive device to personalize to the patient's hemodynamic system.
Specialized delivery catheter with hemodynamic measurement distal and proximal to the implant providing the appropriate readying for adjusting the orifice of a reducer to create the appropriate hemodynamic gradient.
An active and passive adjustment mechanism around the restriction.
The catheter can be equipped with active sensors; the sensors can display ECG information to determine the heart function's characteristics during the cardiac cycle. For example, the analysis can determine the cardiac P wave, QRS complex, and the ST segment to understand the patient's heart condition. Another sensor can also be placed within the vasculature or one of the heart chambers to determine the effect of the pressures in these areas of the heart and how a reduction is caused by the balloon catheter occluding the coronary sinus.

Various modifications and additions can be made without departing from the spirit and scope of this disclosure. Features of each of the various embodiments described above may be combined with features of other described embodiments as appropriate to provide a multiplicity of feature combinations in associated new embodiments. Furthermore, while the foregoing describes several separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example and not otherwise limiting the scope of this disclosure.

Exemplary embodiments have been disclosed above and illustrated in the accompanying drawings. It will be understood by those skilled in the art that various changes, omissions, and additions may be made to that which is specifically disclosed herein without departing from the spirit and scope of the present disclosure.

1. A vascular pressure differential diagnostic system, comprising:
a catheter having proximal and distal ends, the distal end configured for positioning within a patient's vasculature at a pressure monitoring site;
a variable flow restrictor disposed adjacent the distal end of the catheter;
a first pressure sensor disposed distally with respect to the variable flow restrictor; and
a second pressure sensor disposed proximally with respect to the variable flow restrictor, whereby a pressure differential between the first pressure sensor and second pressure sensor is measurable and mappable to varying flow restrictions.

2. The vascular pressure differential diagnostic system of claim 1, further comprising:
a sheath configured to surround and guide the catheter to the monitoring site;
a third pressure sensor disposed at a distal end of the sheath.

3. The vascular pressure differential diagnostic system of claim 1, further comprising a guidewire configured to be received in a guidewire lumen defined by the catheter with an open distal end.

4. The vascular pressure differential diagnostic system of claim 1, wherein:
the catheter comprises a catheter body defining at least one inflation lumen; and
the variable flow restrictor comprises at least one inflatable balloon disposed adjacent the distal end of the catheter body and communicating with the at least one inflation lumen.

5. The vascular pressure differential diagnostic system of claim 4, further comprising means for adjusting the size of a flow restriction created by the variable flow restrictor.

6. The vascular pressure differential diagnostic system of claim 4, wherein:
the variable flow restrictor further comprises at least a first smaller balloon disposed inside at least a second larger balloon; and
the catheter body defines separate inflation lumens for each said balloon.

7. The vascular pressure differential diagnostic system of claim 6, wherein the first and second balloons are disposed eccentrically around the catheter body.

8. The vascular pressure differential diagnostic system of claim 6, wherein:
the first inner balloon comprises a non-resilient fixed diameter balloon; and
the second outer balloon comprises a resilient variable diameter balloon.

9. The vascular pressure differential diagnostic system of claim 4, further comprising a side port formed in the catheter and communicating with the guidewire lumen proximally with respect to said at least one balloon, whereby a variable flow passage through the guidewire lumen is controllable by selectively positioning a stylet or the guidewire over the side port.

10. The vascular pressure differential diagnostic system of claim 4, wherein the balloon comprises an annular balloon with an internal hourglass shape defining a variable flow orifice and an outer periphery configured to contact the vessel wall when inflated.

11. The vascular pressure differential diagnostic system of claim 10, wherein:
the annular balloon has an outer wall formed of a non-resilient material and sized to engage the vascular wall when inflated; and
the annular balloon has an inner wall formed of a resilient material configured to vary the orifice diameter in response to varying inflation pressure.

12. The vascular pressure differential diagnostic system of claim 10, further comprising:
a cinch disposed inside the annular balloon surrounding the variable flow orifice;
a control wire extending through the balloon inflation lumen to the catheter proximal end, the control wire operatively connected to the cinch whereby the cinch may be opened or closed to reduce or increase the variable flow orifice.

13. The vascular pressure differential diagnostic system of claim 4, wherein:
the balloon is configured to fully occlude the vascular lumen when inflated; and
the catheter body defines a variable flow passage through the balloon having an entry port at the catheter distal end and an exit port at a proximal end of the balloon.

14. The vascular pressure differential diagnostic system of claim 13, wherein the variable flow passages comprise a stylet moveable within the variable flow passage to variably obstruct the exit port.

15. The vascular pressure differential diagnostic system of claim 14, wherein:
the catheter body defines a stylet lumen communicating with the variable flow passage and extending to a proximal end of the catheter body; and
the stylet extends through the stylet lumen into the variable flow passage and is manipulable at the proximal end of the catheter body.

16. The vascular pressure differential diagnostic system of claim 4, further comprising:
a hub at the proximal end of the catheter configured to control sheath movement, balloon inflation and guidewire or stylet positioning; and
a control unit in communication with the hub configured to receive information indicating measured pressures and size of flow restriction and to generate a pressure differential map across a plurality of flow restriction sizes.

17. The vascular pressure differential diagnostic system of claim 16, further comprising a cardiac status monitor in communication with the control unit, and wherein the control unit is further configured to map cardiac status as determined by the cardiac status monitor with said pressure differential map across a plurality of flow restriction sizes.

18. A method for determining hemodynamic and cardiac response to vascular flow restriction, comprising:
measuring a base line pressure at a monitoring site within a patient's vasculature;
partially occluding the vascular lumen at the monitoring site with a plurality of differently sized flow restrictions;
measuring a pressure differential across the partial occlusion for each differently sized flow restriction; and
identifying a flow restriction size corresponding to a desired pressure differential.

19. The method of claim 18, further comprising creating a map of measured pressure differential to flow restriction size for a plurality of different sized flow restrictions.

20. The method of claim 18, further comprising:
positioning a pressure differential measuring device at the monitoring site, said measuring device comprising a variable flow restriction and first and second pressure sensors disposed upstream and downstream, respectively, from the variable flow restriction;
partially occluding the vascular lumen and measuring the pressure differential across the partial occlusion using said pressure differential measuring device; and
withdrawing the pressure differential measuring device.

21. The method of claim 18, further comprising correlating changes in a monitored cardiac status indicator with changes in the flow restriction size.

22. The method of claim 21, wherein the monitored cardiac status indicator comprises ST segment elevation.

23. The method of claim 21, further comprising identifying a flow restriction area corresponding to reduction or elimination of ST segment elevation as the flow restriction area for a coronary sinus reducer implant.

24. The method of claim 21, wherein the monitored cardiac status indicator comprises cardiac output.

25. A method for determining hemodynamic and cardiac response to vascular flow restriction, comprising:
positioning a vascular pressure differential diagnostic catheter within a vascular lumen at a monitoring site;
partially occluding the vascular lumen to create a first size flow restriction at the monitoring site with the vascular pressure differential diagnostic catheter;
measuring a pressure differential across the partial occlusion using pressure sensors disposed on the vascular pressure differential diagnostic catheter upstream and downstream from the partial occlusion;
repeating the partially occluding and measuring steps with at least a second size flow restriction, the measuring step performed for each different sized flow restriction;
monitoring patient physiological response to each different sized flow restriction of the partial occlusion;
evaluating patient physiological response to each different sized flow restriction based on predetermined physiological response criteria;
selecting as a candidate for an implanted flow restricting device the flow restriction size corresponding to the predetermined physiological response criteria; and
identifying the flow restriction size corresponding to a desired pressure differential.

26. The method of claim 25, wherein said positioning comprises positioning the vascular pressure differential diagnostic catheter within a patient's coronary sinus.

27. The method of claim 25, wherein the predetermined physiological response criteria comprise a predetermined reduction in elevation of a patient ST segment measured concurrently with the partially occluding.

28. The method of claim 25, wherein the predetermined physiological response criteria comprise a predetermined change in cardiac output measured concurrently with the partially occluding.

29. The method of claim 28, wherein cardiac output is measured by one or more of intracardiac sensors, nuclear perfusion scanning, and ultrasound.

30. The method claim 25, wherein the predetermined physiological response criteria comprise or further comprise a statement of reduced symptoms by an awake patient.

31. The method of claim 27, wherein the reduced symptoms comprise a reduction in angina-related pain.
